(19) United States
(12) Patent Application Publication
(10) Pub. No.: US 2010/0204617 A1
(43) Pub. Date: Aug. 12, 2010

(54) Ultrasonic probe with acoustic output sensing

(76) Inventor: Shmuel Ben-Ezra, Pardes-Hanna (IL)

Correspondence Address: FENNEMORE CRAIG, 3003 NORTH CENTRAL AVENUE, SUITE 2600, PHOENIX, AZ, 85012, US

(21) Appl. No.: 12/379,072

(22) Filed: Feb. 12, 2009

(51) Int. Cl. A61N 7/00; A61B 8/14

(52) U.S. Cl. 601/2; 600/459

(57) ABSTRACT

An ultrasonic probe comprising a source adapted to output ultrasonic energy, and an acoustic output sensor adapted to sense at least a portion of the ultrasonic energy. The ultrasonic probe may be, for example, part of an ultrasound system further comprising a controller, wherein the acoustic output sensor may be adapted to transmit a signal in response to sensed ultrasonic energy.

### FIELD OF THE DISCLOSURE

Embodiments of the disclosure relate to sensing of acoustic output of an ultrasonic probe.

### BACKGROUND

Ultrasound energy, in the form of acoustic waves, is widely used in the medical field, both in diagnostics and in treatment. Therapeutic ultrasound is used for procedures such as ablation and/or destroying of pathogenic objects and various tissues. Ultrasound may also be used in aesthetic medicine to destroy fat and/or cellulite tissues, such as in non-invasive body contouring procedures. The non-invasive body contouring procedure is based on the application of focused acoustic energy that selectively targets and disrupts fat cells, essentially without damaging neighboring structures. This may be achieved, for example, by an ultrasonic probe that delivers focused ultrasound energy to the subcutaneous fat layer. In order to enhance utilization of the probe and thus enhance the efficiency of the body contouring procedure, an assessment of the actual acoustic energy delivered by the probe during operation may be advantageous.

Assessment of energy delivered by ultrasonic probes was previously disclosed, for example, in U.S. Pat. No. 4,625,542, which discloses a radiation power measuring apparatus; U.S. Pat. No. 6,790,180, which discloses measuring power output of an ultrasound transducer; and U.S. Pat. No. 6,016,704, which discloses an electro mechanic transducer with integrated pressure sensor.

### SUMMARY

There is provided, according to an embodiment, an ultrasonic probe comprising a source adapted to output ultrasonic energy; and an acoustic output sensor adapted to sense at least a portion of said ultrasonic energy.

There is further provided, according to an embodiment, an ultrasound system comprising a controller; and an ultrasonic probe comprising a source adapted to output ultrasonic energy and an acoustic output sensor adapted to transmit a signal in response to sensed ultrasonic energy.

In some embodiments, said ultrasonic probe is adapted for use in a therapeutic procedure.

In some embodiments, said therapeutic procedure is a non-invasive body contouring procedure.

In some embodiments, wherein said source comprises a piezoelectric transducer.

In some embodiments, said piezoelectric transducer is selected from a group consisting of: a lead-titanate (PbT) transducer, a lead zirconate titanate (PZT) transducer and a polyvinylidene-fluoride (PVDF) transducer.

In some embodiments, said source comprises an electromagnetic shockwave emitter (EMSE), and further comprising a reflector adapted to focus shockwaves emitted by said EMSE.

In some embodiments, said source comprises a Langevin transducer.

In some embodiments, said acoustic output sensor comprises a piezoelectric transducer.

In some embodiments, said piezoelectric transducer is selected from a group consisting of: a lead-titanate (PbT) transducer, a lead zirconate titanate (PZT) transducer and a polyvinylidene-fluoride (PVDF) transducer.

In some embodiments, said PVDF transducer comprises a PVDF sheet sandwiched between two plastic sheets.

In some embodiments, said acoustic output sensor comprises a fiber optic hydrophone.

In some embodiments, said acoustic output sensor comprises a temperature sensor.

In some embodiments, said source comprises a phased array of piezoelectric elements, and wherein said acoustic output sensor comprises at least one piezoelectric element of said phased array.

In some embodiments, said source comprises a spherical shell section PZT transducer comprising a central hole, and wherein said acoustic output sensor is approximately aligned with said central hole.

In some embodiments, the ultrasonic probe further comprises an indicator adapted to indicate sensed ultrasonic energy.

In some embodiments, said indicator comprises a light emitting diode (LED).

In some embodiments, said indicator comprises a buzzer.

In some embodiments, said controller is adapted to receive said signal and to automatically adjust at least one ultrasonic energy parameter based on said signal.

In some embodiments, said controller is adapted to receive said signal and to indicate sensed ultrasonic energy to an operator of said ultrasound system, to enable said operator to manually adjust at least one ultrasonic energy parameter.

In some embodiments, said ultrasound system is adapted for use in a non-invasive body contouring procedure.

In some embodiments, said controller comprises an input module, a processing module and an output module.

In some embodiments, said source and said acoustic output sensor are both embodied in a same piezoelectric transducer; and said controller is adapted to operate said piezoelectric transducer in a test mode, wherein said source is ordered to output said ultrasonic energy and, consecutively, sense an echo of said ultrasonic energy.

In some embodiments, said controller is adapted to operate said source and said acoustic output sensor in a test mode, wherein said source is ordered to output said ultrasonic energy and, consecutively, said acoustic output sensor senses an echo of said ultrasonic energy.

There is further provided, according to an embodiment, a method for testing an ultrasonic probe of an ultrasound system, the method comprising activating an ultrasonic probe; receiving indication of ultrasonic energy emitted from the ultrasonic probe, wherein the indication is based on a signal transmitted by an acoustic output sensor of the ultrasonic probe; adjusting, based on the indication, at least one ultrasonic energy parameter; and performing treatment using the ultrasound system with the ultrasonic energy parameter set.

In some embodiments, the indication is performed directly by the ultrasonic probe, and comprises at least one indication selected from a group consisting of: a lit light emitting diode (LED), a color-changed sticker and an activated buzzer.

In some embodiments, the indication is performed by a controller of the ultrasound system, and comprises at least one indication selected from a group consisting of: a visual indication and an audible indication.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1 shows a block diagram of an ultrasound system;

FIG. 2 shows a block diagram of an ultrasonic probe;

FIG. 3 shows a cross-sectional view of an ultrasonic probe;

FIG. 4 shows a cross-sectional view of an ultrasonic probe with a polyvinylidene-fluoride (PVDF) acoustic output sensor;

FIG. 5 shows a cross-sectional view of an ultrasonic probe with a fiber optic hydrophone;

FIG. 6 shows a cross-sectional view of an ultrasonic probe adapted for self-detection of echo; and

FIG. 7 shows a cross-sectional view of an ultrasonic probe with a phased array of transducers.

### DETAILED DESCRIPTION

An aspect of some embodiments relates to an ultrasonic probe adapted to provide acoustic energy, wherein the ultrasonic probe is associated with an acoustic output sensor for providing assessment of the actual acoustic output of the probe (as opposed to its theoretical acoustic output which may sometimes not be realized).

The acoustic output sensor may be integrated with the probe, transiently associated with the probe, or permanently associated with it. The acoustic output sensor may include any type of sensor which is adapted to directly or indirectly sense an actual acoustic energy delivered by the probe. The acoustic output sensor may include one or more sensors of one or more types. For example, the acoustic output sensor may be adapted to sense the pressure created by the acoustic energy emitted from the ultrasonic probe. The acoustic output sensor may be adapted to measure/detect all or any portion of the acoustic energy emitted from the probe. The acoustic-output sensor may be further adapted to provide a feedback signal related to the operation of the probe. The feedback signal may be provided, for example, to a controller, that may accordingly adjust the operation of the ultrasonic probe such that operation of the ultrasonic probe is optimally performed. The feedback signal may be provided, for example, to an operator of the probe, for example by visual, audible and/or any other means.

### Glossary

The term “sense”, as referred to herein in regard to an acoustic output sensor, may refer to detection and/or measurement of acoustic energy emitted from an ultrasonic probe. Whereas “detection” may refer to a binary determination of the existence or non-existence of acoustic energy, “measurement” may refer to a quantitative determination of an amount or level of acoustic energy.

The terms “acoustic energy/power” and “ultrasonic energy/power”, as referred to herein in regard to an ultrasonic probe, may refer to ultrasonic waves emitted from an ultrasonic probe, waves which are often expressed by detectible and/or measurable pressure changes.

The terms “treated subject” or simply “subject”, as referred to herein, may refer to a patient being treated with a non-invasive body contouring procedure.

The term “operator”, as referred to herein, may refer to any person operating an ultrasound system. The operator may be a caregiver such as a physician, a nurse or an otherwise qualified person.

Reference is now made to FIG. 1, which shows a block diagram of an ultrasound system (100). Ultrasound system (100) may include the following units: a controller (101), an ultrasonic probe (hereinafter “probe”) (102) and an interconnecting cable (not shown) in between.

Controller (101) may include the following modules: an input module (104), an output module (105) and a processing module (106).

Input module (104) may be adapted to collect information from an operator. For this purpose, input module (104) may employ one or more control devices (not shown) such as keyboards, push-buttons, knobs, sliders and/or the like. In addition, input module (104) may be connected to at least one microphone for receiving voice commands spoken by the operator.

Input module (104) may be adapted to collect information from and/or to interact with one or more peripheral devices. It may use a camera, for example, to track a position of a treated subject. It may use a thermometer, for example, to detect environmental conditions along the treatment. It may be connected to one or more communication networks like the internet, an intranet, a cellular phone network and the like.

Input module (104) may be adapted to collect information from probe (102). For example, it may receive a temperature value measured inside probe (102). As another example, it may receive digital information stored in probe (102), such as information pertaining to its operation frequency.

Output module (105) may supply the electric energy required to drive probe (102), by outputting electric signals, electric currents, and/or the like. Output module (105) may further supply probe (102) with a flow of a cooling liquid, or the like, if such is required for the operation of the probe.

Output module (105) may provide the operator with information regarding the treatment. For this purpose, output module (105) may display messages and/or graphics on one or more monitors. Output module (105) may switch on and off lights, LEDs (light emitting diodes), and/or the like, to indicate one or more parameters to the operator. In addition, output module (105) may use audible messages, tones and/or the like.

Output module (105) may send and/or receive information through a communication network like the internet, an intranet, a cellular phone network and the like.

Processing module (106) may control the operation of the controller (101) by processing data provided by input module (104) and, accordingly, configuring output module (105).

Probe (102) may produce ultrasound energy. In systems designed for therapeutic and/or aesthetic ultrasound treatments, the probe may be adapted to deliver ultrasound energy into a body of a subject. The ultrasound energy delivered by the probe may be focused, thus reaching high intensities at a focal zone. Ultrasound system (100) may contain more than one probe connected to controller (101).

Probe (102) may be handheld by the operator. The operator, in this case, may manually position probe (102) in a position relative to the body of the treated subject. Additionally or alternatively, probe (102) may be carried by a mechanical arm. The mechanical arm, in this case, may place the probe (102) in a position relative to the body of the subject. Probe (102) may be carried by a combination of a mechanical arm and the operator.

Probe (102) may be a replaceable part of the system (100), thus enabling the substitution of one probe with another one of the same or a different type. Ultrasound system (100) may be designed to operate with more than one type of a probe.

A cable (not shown) may form a mechanical connection between controller (101) and probe (102). It may be an integral part of probe (102) or controller (101). It may transmit electrical currents, signals, fluids and/or digital data from controller (101) to probe (102) and from the probe to the controller.

In an embodiment, probe (102) is advantageously adapted to provide input module (104) with information related to an acoustic energy that is generated by probe (102). The information may include a binary determination of the existence or non-existence of acoustic energy, and/or a measurement of an amount or level of acoustic energy generated by probe (102).

The information may be processed by processing module (106) and consequently, output module (105) may modify one or more parameters pertaining to an electric power delivered to probe (102). Additionally or alternatively, output module (105) may indicate the information to the operator by visual means (such as a message on a monitor, a light and/or the like), audible means (audible messages, tones and/or the like) and/or other means. The operator may choose to manually modify one or more parameters pertaining to an electric power delivered to probe (102), responsive to the indicated information.

In an embodiment, the information regarding the acoustic output that is generated by probe (102) is indicated directly to the operator, without having to be funneled through any module of controller (101). For this purpose, probe (102) may be equipped with an indicator—a device adapted to generate a visible indication (a light using a LED or any other lighting device, a color-changing sticker on the probe, a message on a small screen installed on the probe itself and/or the like), an audible indication (a tone, a verbal message and/or the like issued by a buzzer or a speaker), and/or the like.

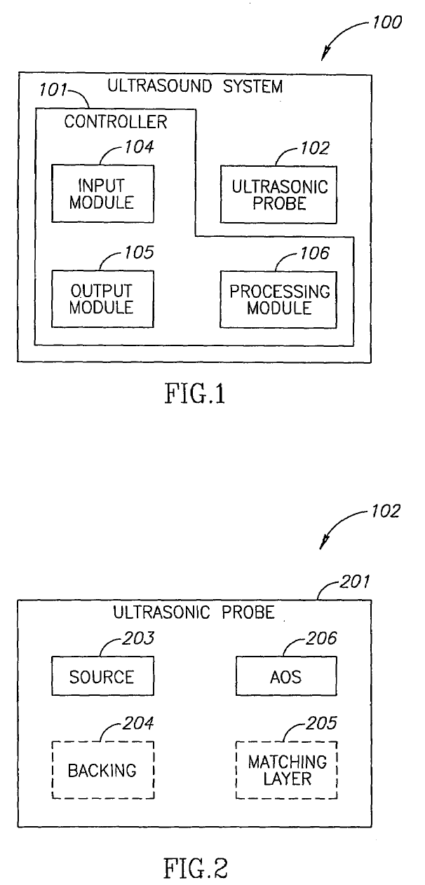

Reference is now made to FIG. 2, which shows a more detailed block diagram of probe (102) of FIG. 1. Probe (102) may include a case (201), a source (203), an acoustic output sensor (hereinafter “AOS”) (206) and, optionally, a backing (204) and/or matching layer (205). Probe (102) may be assembled inside case (201), which may be made of metal (aluminum or the like), plastic and/or any other suitable material. If the probe is designed to be manually held during treatment by the operator, the design of case (201) may comply with ergonomic considerations. If probe (102) is designed to be carried during treatment by a mechanical arm, case (201) may be adapted to be mounted to the mechanical arm, such as using a dedicated connector.

Source (203) may be adapted to generate ultrasonic energy, optionally delivered in the form of a beam, traveling from probe (102) into the treated subject. Source (203) may convert (or “transduce”) electrical power to acoustic power. The acoustic energy may be generated in shock-waves, pulses, bursts, a continuous wave and/or in any other form. Source (203) may be realized by a piezoelectric material capable of converting electrical energy into ultrasonic energy (and vice versa). Among piezoelectric materials that may be used to realize source (203) are lead-titanate (PbT), lead zirconate titanate (PZT) and polyvinylidene-fluoride (PVDF).

Source (203) may be made of a single piezoelectric element or multiple elements. The geometric shape of the source (203) may be flat, spherical shell section, cylindrical shell section, or any other shape. Source (203) may be a phased-array source, constructed from multiple piezoelectric elements, wherein each element may be excited separately, in a defined sequence, in order to generate an ultrasound beam of special characteristics, such as a focused beam, a diverging beam, or a beam having other special characteristics.

Source (203) may be realized by a combination of one or more piezoelectric elements coupled to one or more resonators—solid blocks of metal or another material adapted to resonate upon excitation of the piezoelectric elements. A Langevin transducer is an example known in the art for such a combination, where a piezoelectric element is “sandwiched” between two metal pistons serving as resonators.

Source (203) may be realized by an electromagnetic shock-wave emitter (hereinafter “EMSE”), a technology that is known in the art and is being used, for example, in extra-corporeal shockwave lithotripsy.

Probe (102) may optionally include backing (204). Backing (204) may be a reflector and/or an absorber. If a reflector, backing (204) may reflect a portion of ultrasonic energy that is radiated from source (203) backwards and direct it forward, in the direction of the treated subject. In an absorber, backing (204) may absorb a portion of ultrasonic energy that is radiated from the source (203) backwards.

Probe (102) may optionally include a matching layer (205) adapted to physically conduct the acoustic energy from source (203) to a skin surface of the treated subject, as well as towards AOS (206) that is described below. Matching layer (205) may be a material in a solid, a fluid and/or a gel form. Matching layer (205) may be realized from more than one component; for example, it may include several layers of different polymers. Matching layer (205) may be made, for example, from liquid oil contained within a plastic sheet. The material of matching layer (205) may provide electrical insulation (or at least a very low conductivity) and/or good thermal conductivity.

In an embodiment, probe (102) is advantageously provided with AOS (206). AOS (206) may be adapted to provide a signal in response to acoustic power, pressure, pressure variation, temperature elevation and/or any physical change caused by the acoustic power emitted by source (203). The signal provided by AOS may be electrical, optical and/or any other type of signal. Furthermore, the signal may be a detection signal and/or a measurement signal: A detection signal may include a binary indication of the existence or non-existence of acoustic energy, namely—a simple indication of the on or off status of probe (102), for determining proper functioning of the probe. A measurement signal may include a quantitative indication of an amount or level of acoustic energy, measured in any units known in the art.

AOS (206) may be confined to a small region in probe (102), may be distributed over large regions in the probe, and may include one or more devices of the same type or different types. AOS (206) may be located anywhere inside or outside case (201). For example, if AOS (206) is located inside case (201), it may be positioned inside, above, below or around source (203), inside or behind optional backing (204), and/or inside optional matching layer (205).

AOS (206) may be realized as an integral part of source (203). AOS (206) may be realized as an integral part of probe (102), permanently attached to the probe or transiently attached to it.

AOS (206) may be realized by a conventional hydrophone available on the market, such as, for example, a fiber optic hydrophone of model no. FOPH 2000, available from RP Acoustics e.K., Germany. AOS (206) may be realized by one or more elements made of a piezoelectric material, such as a conventional piezoelectric hydrophone of model no. HNR-500 available from ONDA Corp., Sunnyvale, Calif., USA. AOS (206) may be realized by an optical device such as a fiber-optic hydrophone adapted to detect a change in optical characteristics of a material, induced by the acoustic power emitted by source (203).

AOS (206) may be realized by a temperature sensor such as a thermistor, adapted to detect and/or measure temperature and/or variations in temperature induced by the acoustic power emitted by source (203).

AOS (206) may be realized by multiple elements of any combination of the above described examples.

The shape, size and location of AOS (206) as part of the probe (102) may be subject to a specific design of the probe and the AOS. A number of examples are given below and shown in FIGS. 3-7. However, those of skill in the art will recognize that a probe having an AOS may be realized in many other configurations.

Reference is now made to FIG. 3, which shows a cross-sectional view of an exemplary ultrasonic probe (hereinafter “probe”) (300). A case (301) of probe (300) may be made of aluminum having a cylindrical shape. Case (301) may include a circular window (307) in its lower part, being, essentially, a hole in the aluminum case. A source (303) may be a single piezoelectric element made of PZT. The shape of source (303) may be a spherical shell section having a uniform thickness. Source (303) may include a round hole (303a) in its center. Optionally, a backing (304) may be provided above source (303).

An AOS (306) may be a piezoelectric element made of PZT, in the shape of a circular disk. AOS (306) may be horizontally aligned with hole (303a) and vertically positioned above it or at least partially inside it. A matching layer (305) may be made of polyurethane and may homogeneously fill the space around source (303) and around AOS (306). The polyurethane may also fill circular window (307) at the lower part of case (301), optionally slightly protruding from the case, so as to form a physical interface with the treated subject's skin surface.

Upon activation of probe (300), source (303) may receive electric power from the controller (not shown) and convert it into ultrasonic power. At least some portion of the generated ultrasonic power may reach AOS (306), either directly or by reflections reaching from various parts of probe (300). AOS (306) may, in turn, convert it to an electric signal. The electric signal of AOS (306) may be acquired and processed by the controller, which may automatically adjust one or more parameters of electric activation of the probe, or may enable the operator to change these parameters manually. For example, a parameter such as an electrical power or voltage may be increased if the measured output power of source (303) is lower than desired, and vice versa. Additionally or alternatively, the electric signal generated by AOS (306) may directly activate a LED (308) located on case (301). The activated LED (308) is visible to the operator. In other embodiments (not shown), the electric signal generated by an AOS may directly activate a buzzer and/or a speaker located on a probe's case, and/or may cause a sticker attached to the case to change its color.

In other embodiments (not shown), an AOS may be located below a source. In yet further embodiments (not shown), an AOS may be located inside a probe's backing, especially if the backing is of the absorbent type, since acoustic energy is usually weaker inside the absorbent and, therefore, the AOS may incur less wear.

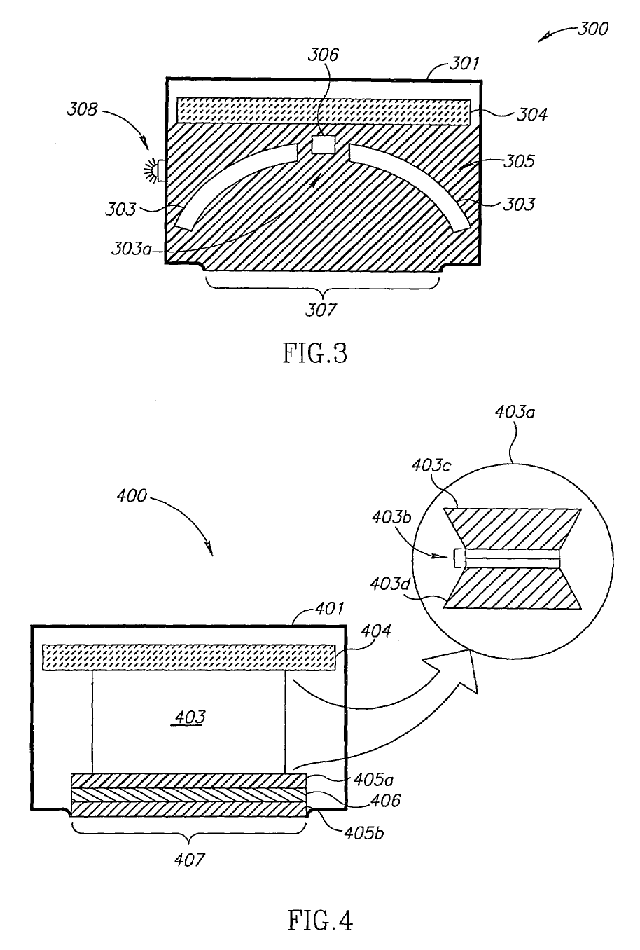

The following embodiments, described in connection to FIGS. 4-7, may not include a description of the indication of detected/measured acoustic power and adjustment of parameters, accordingly. Those of skills in the art will recognize that what is described here regarding FIG. 3 may similarly be applied to the embodiments of FIGS. 4-7.

Reference is now made to FIG. 4, which shows a cross-sectional view of an ultrasonic probe (hereinafter “probe”) (400) with a PVDF AOS (406). A case (401), a circular window (407), and/or an optional backing (404) which includes an absorbent material, may be similar to those shown in FIG. 3.

A source (403) may be a Langevin transducer, shown with more detail at (403a). The Langevin transducer may be constructed from one or more PZT disks (403b) sandwiched between two aluminum pistons (403c-d). The design of a matching layer may contain AOS (406) within it. The matching layer may include three layers: A first layer may be a plastic sheet (405a) adhered to source (403); a second layer may be AOS (406) which is made of a PVDF sheet, and a third layer may be of another plastic sheet (405b).

In other embodiments (not shown), an AOS may be made of a PVDF sheet and is located behind an absorber. In this design, the AOS may detect the residual acoustic power that still propagates beyond the absorber.

In other embodiments (not shown), an AOS is made of a PVDF sheet and is located inside an absorber. In this design, the AOS may detect the residual acoustic power that managed to reach it.

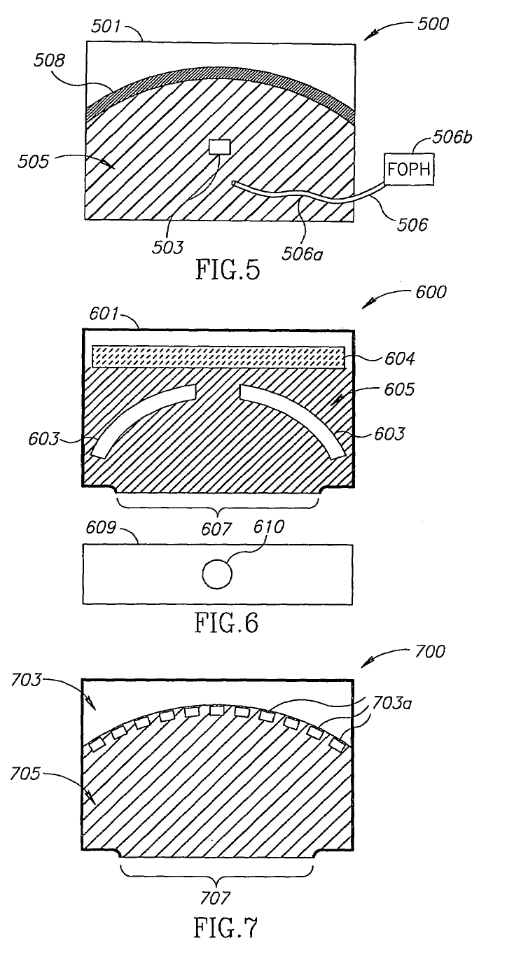

Reference is now made to FIG. 5, which shows a cross-sectional view of an ultrasonic probe (hereinafter “probe”) (500) with a fiber-optic hydrophone (506) serving as an AOS, and an electromagnetic shock-wave emitter (EMSE) serving as a source (503).

Case (501) of probe (500) may be similar to that of FIG. 3, but may have different measurements.

Source (503) may generate a shockwave by an electromagnetic discharge of capacitors. The shockwave may propagate in a matching layer (505), which may include oil or any other liquid of suitable properties, such as density, compressibility, electrical resistivity, thermal conductivity, speed of sound and/or the like. A concave reflector (508) may focus the shockwave into a focal zone, generally located inside the body of a subject. AOS (506) may be realized by a fiber-optic hydrophone (506a) located in matching layer (505), connected to an apparatus (506b) having the required electro-optics and electronics for performing pressure measurement and displaying and/or transmitting a result. Apparatus (506b) may display to the operator a pressure signal acquired by fiber-optic hydrophone (506a). AOS (506) may be permanently or transiently attached to probe (500). Transient attachment may enable replacing AOS (506) in the same probe, and/or using the same AOS for more than one probe.

Reference is now made to FIG. 6, which shows a cross-sectional view of an ultrasonic probe (hereinafter “probe”) (600) adapted for self-detection of echo.

A case (601), a circular window (607), a matching layer (605) and/or an optional backing (604), which includes an absorbent material, may be similar to those shown in FIG. 3.

A source (603) may be made of a single piezoelectric element, optionally similar to that of source (303) of FIG. 3. Source (603) itself may function as an AOS by receiving reflections of its own acoustic emissions. Source (603) may convert the reflected ultrasonic power into electrical power and provide a controller (not shown) with a suitable signal. Reflection of ultrasonic power back to source (603) may occur, for example, from a surface of the probe's case (601).

A process of acoustic output detection and/or measurement may be combined into a treatment and/or be performed in a separate process of acoustic output measurement carried out before, after or at an intermission of the treatment.

If the process is combined into a treatment, the operator may receive continuous or intermittent notifications as to the acoustic output of probe (600).

If the process is performed separately, the operator may detach probe (600) from the skin surface of the treated subject and place it in the open air. By this, strong reflections are likely to occur from the interface between the probe and the air. For the purpose of this process, the controller may include a “test mode”, in which the controller may provide electric power that is suitable for echo detection. For example, the electric power may be delivered as an “excitation signal” of a very short pulse (commonly having duration of approximately 1 microsecond). The echo signal that is received in the controller is, naturally, delayed in time and, thus, distinguishable from the excitation signal. The time delay between excitation signal and echo signal is defined as T=L/C, where C is the velocity of sound in matching layer (605) and L is the length of the trajectory passed by the ultrasonic energy.

In another embodiment, a measurement fixture (609) is used as a reflector. The operator may place probe (600) on fixture (609) in order to perform acoustic output measurement—before, after, or during an intermission of the treatment. Fixture (609) may be made of a material adapted to allow the passage of ultrasonic energy from probe (600) with minimal reflections. An exemplary suitable material for the fixture is polyurethane. Fixture (609) may include a reflector (610) positioned in a specific and known distance from source (603), such as at approximately the focal point of the curvature of source (603). Reflector (610) may be realized, for example, by a metal ball, an air bubble or any other object adapted to efficiently reflect acoustic waves. The material, shape and/or size of reflector (610) may be suited for properties of the pertinent ultrasonic energy (such as its frequency and/or the like) and, optionally, for properties of the material of fixture (609).

Reference is now made to FIG. 7, which shows a cross-sectional view of an ultrasonic probe (hereinafter “probe”) (700) with a phased array of transducers serving as a source (703), and a matching layer (705) adjacent to the source. Similar to the embodiment of FIG. 6, the source here also serves as an AOS.

For example, source (703) may include multiple piezoelectric elements, such as elements (703a), made of PZT, wherein each of the elements may be excited individually by a controller (not shown). In this embodiment, source (703) may function also as an AOS. The controller may shift into a “measurement mode”, at the operator's command or automatically. In “measurement mode”, one, a group of, or all the piezoelectric elements may be sequentially tested. A tested element or a group of tested elements are individually or jointly, respectively, excited by the controller. Consequently, the tested element(s) generate(s) ultrasonic power. Some portion of the generated ultrasonic power is received by one or more neighboring piezoelectric element, either directly or by virtue of reflections. The neighboring element(s) may convert the detected ultrasonic power into electric signals. The controller may receive the signals from the neighboring element(s) and process them in order to evaluate the status of the tested element(s). For the evaluation of the tested element(s), the controller may process the electric signals received from the one or more other elements, up to the entirety of the existing elements.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

In the description and claims of the application, each of the words “comprise” “include” and “have”, and forms thereof, are not necessarily limited to members in a list with which the words may be associated.

What is claimed is:

1. An ultrasonic probe comprising:
a source adapted to output ultrasonic energy; and
an acoustic output sensor adapted to sense at least a portion of said ultrasonic energy.

2. The ultrasonic probe according to claim 1, wherein said ultrasonic probe is adapted for use in a therapeutic procedure.

3. The ultrasonic probe according to claim 2, wherein said therapeutic procedure is a non-invasive body contouring procedure.

4. The ultrasonic probe according to claim 1, wherein said source comprises a piezoelectric transducer.

5. The ultrasonic probe according to claim 4, wherein said piezoelectric transducer is selected from a group consisting of: a lead-titanate (PbT) transducer, a lead zirconate titanate (PZT) transducer and a polyvinylidene-fluoride (PVDF) transducer.

6. The ultrasonic probe according to claim 1, wherein said source comprises an electromagnetic shockwave emitter (EMSE), and further comprising a reflector adapted to focus shockwaves emitted by said EMSE.

7. The ultrasonic probe according to claim 1, wherein said source comprises a Langevin transducer.

8. The ultrasonic probe according to claim 1, wherein said acoustic output sensor comprises a piezoelectric transducer.

9. The ultrasonic probe according to claim 8, wherein said piezoelectric transducer is selected from a group consisting of: a lead-titanate (PbT) transducer, a lead zirconate titanate (PZT) transducer and a polyvinylidene-fluoride (PVDF) transducer.

10. The ultrasonic probe according to claim 9, wherein said PVDF transducer comprises a PVDF sheet sandwiched between two plastic sheets.

11. The ultrasonic probe according to claim 1, wherein said acoustic output sensor comprises a fiber optic hydrophone.

12. The ultrasonic probe according to claim 1, wherein said acoustic output sensor comprises a temperature sensor.

13. The ultrasonic probe according to claim 1, wherein said source comprises a phased array of piezoelectric elements, and wherein said acoustic output sensor comprises at least one piezoelectric element of said phased array.

14. The ultrasonic probe according to claim 1, wherein said source comprises a spherical shell section PZT transducer comprising a central hole, and wherein said acoustic output sensor is approximately aligned with said central hole.

15. The ultrasonic probe according to claim 1, further comprising an indicator adapted to indicate sensed ultrasonic energy.

16. The ultrasonic probe according to claim 15, wherein said indicator comprises a light emitting diode (LED).

17. The ultrasonic probe according to claim 15, wherein said indicator comprises a buzzer.

18. An ultrasound system comprising:
a controller; and
an ultrasonic probe comprising a source adapted to output ultrasonic energy and an acoustic output sensor adapted to transmit a signal in response to sensed ultrasonic energy.

19. The ultrasound system according to claim 18, wherein said controller is adapted to receive said signal and to automatically adjust at least one ultrasonic energy parameter based on said signal.

20. The ultrasound system according to claim 18, wherein said controller is adapted to receive said signal and to indicate sensed ultrasonic energy to an operator of said ultrasound system, to enable said operator to manually adjust at least one ultrasonic energy parameter.

21. The ultrasound system according to claim 18, wherein said ultrasound system is adapted for use in a non-invasive body contouring procedure.

22. The ultrasound system according to claim 18, wherein said controller comprises an input module, a processing module and an output module.

23. The ultrasound system according to claim 18, wherein said source comprises a piezoelectric transducer.

24. The ultrasound system according to claim 23, wherein said piezoelectric transducer is selected from a group consisting of: a lead-titanate (PbT) transducer, a lead zirconate titanate (PZT) transducer and a polyvinylidene-fluoride (PVDF) transducer.

25. The ultrasound system according to claim 18, wherein said source comprises an electromagnetic shockwave emitter (EMSE), and further comprising a reflector adapted to focus shockwaves emitted by said EMSE.

26. The ultrasound system according to claim 18, wherein said source comprises a Langevin transducer.

27. The ultrasound system according to claim 18, wherein said acoustic output sensor comprises a piezoelectric transducer.

28. The ultrasound system according to claim 27, wherein said piezoelectric transducer is selected from a group consisting of: a lead-titanate (PbT) transducer, a lead zirconate titanate (PZT) transducer and a polyvinylidene-fluoride (PVDF) transducer.

29. The ultrasound system according to claim 28, wherein said PVDF transducer comprises a PVDF sheet sandwiched between two plastic sheets.

30. The ultrasound system according to claim 18, wherein said acoustic output sensor comprises a fiber optic hydrophone.

31. The ultrasound system according to claim 18, wherein said acoustic output sensor comprises a temperature sensor.

32. The ultrasound system according to claim 18, wherein:
said source and said acoustic output sensor are both embodied in a same piezoelectric transducer; and
wherein said controller is adapted to operate said piezoelectric transducer in a test mode, wherein said source is ordered to output said ultrasonic energy and, consecutively, sense an echo of said ultrasonic energy.

33. The ultrasound system according to claim 18, wherein said source comprises multiple piezoelectric elements, and wherein said acoustic output sensor comprises at least one piezoelectric element of said multiple piezoelectric elements.

34. The ultrasound system according to claim 33, wherein said multiple piezo elements constitute a phased array.

35. The ultrasound system according to claim 33, wherein said controller is adapted to operate said source and said acoustic output sensor in a test mode, wherein said source is ordered to output said ultrasonic energy and, consecutively, said acoustic output sensor senses an echo of said ultrasonic energy.

36. The ultrasound system according to claim 18, wherein said source comprises a spherical shell section PZT transducer comprising a central hole, and wherein said acoustic output sensor is approximately aligned with said central hole.

37. The ultrasound system according to claim 18, wherein said ultrasonic probe further comprises an indicator adapted to indicate sensed ultrasonic energy based on said signal.

38. The ultrasound system according to claim 37, wherein said indicator comprises a light emitting diode (LED).

39. The ultrasound system according to claim 37, wherein said indicator comprises a buzzer.

40. A method for testing an ultrasonic probe of an ultrasound system, the method comprising:
activating an ultrasonic probe;
receiving indication of ultrasonic energy emitted from the ultrasonic probe, wherein the indication is based on a signal transmitted by an acoustic output sensor of the ultrasonic probe;
adjusting, based on the indication, at least one ultrasonic energy parameter; and
performing treatment using the ultrasound system with the ultrasonic energy parameter set.

41. The method according to claim 40, wherein the indication is performed directly by the ultrasonic probe, and comprises at least one indication selected from a group consisting of: a lit light emitting diode (LED), a color-changed sticker and an activated buzzer.

42. The method according to claim 40, wherein the indication is performed by a controller of the ultrasound system, and comprises at least one indication selected from a group consisting of: a visual indication and an audible indication.
